(19) United States
(12) Patent Application Publication
(10) Pub. No.: US 2002/0017619 A1
(43) Pub. Date: Feb. 14, 2002

(54) Processing/observing instrument

(76) Inventors: Hiroshi Hirose, Hitachinaka-shi (JP); Tsuyoshi Ohnishi, Hitachinaka-shi (JP); Yoshio Arima, Hitachinaka-shi (JP); Hiroshi Iwamoto, Higashiibaraki-gun (JP)

Correspondence Address: ANTONELLI TERRY STOUT AND KRAUS, SUITE 1800, 1300 NORTH SEVENTEENTH STREET, ARLINGTON, VA, 22209

(21) Appl. No.: 09/531,527

(22) Filed: Mar. 21, 2000

Related U.S. Application Data
(63) Continuation of application No. 09/022,448, filed on Feb. 12, 1998, now abandoned.

(30) Foreign Application Priority Data
Feb. 12, 1997 (JP) 9-027803

(51) Int. Cl. G21G 5/00; A61N 5/00

(52) U.S. Cl. 250/492.3

(57) ABSTRACT

A processing/observing instrument which allows for obtaining a SIM image of a processed cross section of a specimen without changing an angle of the specimen. This processing/observing instrument includes a processing ion beam irradiation system which processes the surface of a specimen with an irradiation of a focused ion beam, an observing ion beam irradiation system which, with an exposure of a focused ion beam, detects secondary ions emitted from the specimen to detect the surface condition of the specimen, a specimen holder which holds the surface of the specimen at a point of intersection of a processing ion beam exposure axis and an observing ion beam exposure axis, and a display which displays the surface condition of the specimen.

### BACKGROUND OF THE INVENTION

The present invention relates to a processing/observing instrument which processes and observes a specimen, using an ion beam.

Semiconductor manufacturing technology has a tendency to employ not only a larger wafer but also finer and more multi-layered wiring. All facilities used to manufacture and check semiconductors have to be developed in accordance with this tendency. For example, a focused ion beam (hereinafter “FIB”) device, which shaves semiconductor material partially and observes the cross section to analyze whether it is defective, is no exception.

Usually, a processing/observing instrument equipped with an FIB comprises a processing unit which processes a specimen using a narrowly focused ion beam, and an observing unit which detects secondary charged particles emitted from the specimen in the case of the ion beam irradiation and produces an SIM (a scanning ion microscope) image. The focused ion beam processes the specimen, usually resulting in a processed hole the cross section of which is parallel to a beam axis of the FIB. In order to observe the cross section of the processed hole by means of a scanning with the focused ion beam, a conventional FIB instrument had to tilt the cross section towards the beam axis (namely, tilt the specimen that has been processed).

In order to tilt the specimen, the conventional cross section processing/observing instrument necessitated a large specimen chamber. This increased the weight of the whole device, thus causing the problem of where to set the device. This problem, as the sample grows bigger, becomes more serious.

FIGS. 3A and 3B show a typical specimen chamber used in a processing/observing arrangement with an FIB. As shown in the cross-sectional side view of FIG. 3B, the specimen chamber, which is surrounded by a plate of h in thickness, is a housing of H in height (the length in the beam irradiation axis direction). Its upper plate is a square roof board 18 shown in FIG. 3A. In the case of processing and observing, a space 17a inside the specimen chamber 17 is maintained under a high vacuum. This causes an atmospheric pressure to press the roof board of the housing, producing a strain as is shown in FIG. 3B. This strain brings about a strain in the beam axis, which prevents the intended performance from being achieved. Accordingly, the strain must be made as small as possible. The value of the strain d, when the material and configuration are left unchanged, is proportional to the fourth power of one side length a of the roof board 18, and is inversely proportional to the third power of the thickness h of the specimen chamber material. This holds true in each plane of the specimen chamber 17.

If, considering the size of the housing, the beam irradiation axis is at the center of the housing, the specimen 7 must be displaced by a distance twice as long as the specimen size in both a vertical and a transverse direction, respectively, in order to irradiate the beam to the whole area of the specimen 7. One side length a of the roof board 18, consequently, must be greater than a value obtained by adding twice the specimen diameter to twice the thickness h of the specimen chamber material. Thus, the cross section area of the specimen chamber 17 perpendicular to the irradiation axis is greater than about 4 times the specimen size, which makes a pressure which is applied by an atmosphere when the inside of the chamber is maintained under vacuum about 4 times greater than would otherwise be the case.

Additionally, when changing an angle of the specimen to the beam irradiation axis into 0° through 90°, the height H of the specimen chamber 17 needs to be made greater than either the sum of the thickness of the stage 6 and that of the specimen 7 or twice the diameter of the specimen 7, depending on which is greater (usually the diameter of the specimen).

From the above mentioned discussion, the larger the specimen diameter grows, the bigger the size of the specimen chamber 17 has to be made and the higher the pressure applied thereto becomes. This shows that, in order to prevent the strain from growing large, the thickness h of the specimen chamber material has to be made thick exponentially.

Taking, as a unit 1, the weight of the specimen chamber 17 accompanied by the specimen 100 mm (4 inches) in diameter, the weight ratios of many sizes of specimen chambers 17 compared to this unit, the material thicknesses h of which are defined so that their strains d will be equal to the strain of this specimen chamber, are shown in FIG. 4.

In the semiconductor manufacturing industry, the diameter of a wafer used has typically from 100 mm through 150 mm to 200 mm. The diameter of the next generation, however, is expected to become 300 mm through 400 mm. FIG. 4 shows the weight ratios of the specimen chambers with sizes which will enable them to support these specimen of the next generation. This figure clearly shows that the weight ratios of the specimen chambers 17 suddenly get large between 200 mm and 300 mm. According to the prior art, consequently, the device that processes and observes a specimen with a diameter greater than 300 mm cannot help becoming extremely heavy, thus resulting in a big problem of where to set it.

Additionally, when repeating the processing and the observing continuously, an angle change (usually some 45° through 60°) of the specimen must be repeated, which has sometimes led to not only a troublesome operation but also a problem of accuracy that the limitation on the position accuracy of the specimen stage makes it difficult to perform the microprocessing of the specimen.

### SUMMARY OF THE INVENTION

It is an object of the present invention to provide a processing/observing instrument which can obtain a SIM image of the processed cross section without changing an angle of the specimen.

The present invention provides a processing/observing instrument which comprises a processing ion beam irradiation system that processes the specimen surface with an irradiation of a focused ion beam, an observing ion beam irradiation system that makes the specimen emit secondary charged particles with an irradiation of a focused ion beam, a specimen holder for holding the specimen surface at a point of intersection of a processing ion beam irradiation axis and an observing ion beam irradiation axis, a detector for detecting the above mentioned secondary charged particles to produce an output of the detection result, and a display for displaying the output from the detector.

The processing/observing instrument embodying the present invention, by incorporating a processing ion column and an observation-only ion column, finds it unnecessary to change an angle of the specimen so as to process and observe it. In other words, there is no need of changing an angle of the specimen stage in the device, which also makes it unnecessary to enlarge the chamber holding the specimen. This means that, even when processing a large-sized specimen, it does not cost too much to process the specimen. In this invention, accordingly, it is preferable that the specimen holder holds the specimen in such a manner that the specimen surface to be processed coincides with a plane, the normal of which is the irradiation axis of the above-mentioned processing ion beam irradiation system.

As mentioned above, according to the prior art, when the specimen size exceeds 300 mm the specimen chamber weight increases extraordinarily. This seriously reduces the practicality of the system. The present invention, however, makes it possible to observe the specimen without tilting it. This, even when the specimen size is larger than 300 mm, makes it possible to reduce the height H of the specimen chamber 17 to not more than 0.3 times that of the specimen chamber used in the conventional device, thus making the whole weight about half as heavy as the conventional device.

It is also preferable that the processing ion beam irradiation system can, by changing lens conditions and mask locations in accordance with external instruction, change the beam between a projection ion beam and the focused ion beam.

The observing ion beam irradiation system, as in the case with the processing system, comprises an ion beam irradiation column, an ion source, and an ion beam focusing unit. This observing ion beam irradiation system can also be formed as a focused ion beam instrument, such as one usable in a range of probe current of 30 pA or less. Additionally, the observing system can also comprise an ion beam deflecting unit.

According to the present invention, it is possible to obtain a SIM image of the processed cross section without changing an angle of the specimen. An increase in the specimen chamber weight accompanied by an increase in the specimen size can be suppressed, which makes the weight of the whole device about half as heavy as that of the conventional device. Also, since the specimen is not tilted, the processing/observing efficiency is improved.

From the above description, it can be seen that a basic concept of the present invention is to use a first ion beam irradiation system for processing and a second ion beam irradiation system, tilted relative to the first ion beam irradiation system, for observing. As noted from the above discussion, this arrangement has several distinct advantages. As a further refinement of this, it is noted that the first ion beam irradiation system which is used for processing can also be used for carrying out a rough observing operation. The second ion beam irradiation system can then be used for carrying out fine observation. For example, the first ion beam irradiation system can be used to carry out a rough observation as an initial step to ensure proper placement of a substrate prior to processing. The first ion beam irradiation system can then proceed with the processing based on the rough observation which it has made. Subsequently, the second ion beam irradiation system can be used to carry out a fine observation of the processed device. To put this another way, the first ion beam processing system can, in fact, have both a processing mode and rough observing mode.

### BRIEF DESCRIPTION OF THE DRAWINGS

FIG. 1 is a diagram illustrating the processing/observing instrument in accordance with a first embodiment of the present invention;

FIG. 2 is a graph illustrating the relationship between a probe diameter and a probe current in each mode of the ion beam systems;

FIG. 3A is a plan view of a roof board of a specimen chamber and FIG. 3B is a cross-sectional view illustrating the strain caused by maintaining the interior of the specimen chamber under vacuum;

FIG. 4 is a graph illustrating the relationship between a wafer diameter and a weight of the specimen chamber in the prior art;

FIG. 5A is a diagram illustrating an operation of the processing ion beam irradiation system of a second embodiment in the projection beam mode and FIG. 5B is a diagram illustrating an operation of the processing ion beam irradiation system of the second embodiment in the scanning beam mode;

FIG. 6 is a diagram illustrating the processing/observing instrument according to a third embodiment;

FIG. 7 is a diagram illustrating the processing/observing instrument according to a fourth embodiment; and

FIG. 8 is a diagram illustrating examples of the images displayed in the fourth embodiment of FIG. 7.

### DESCRIPTION OF THE PREFERRED EMBODIMENTS

The embodiments of the present invention will be described below with reference to the accompanied drawings.

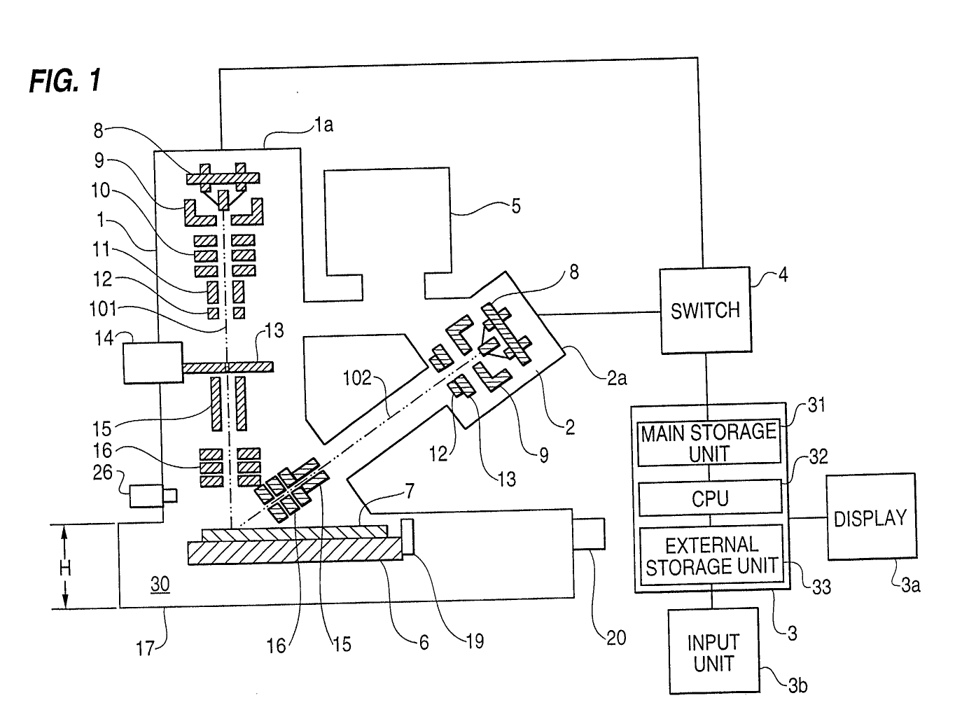

FIG. 1 shows a constitution of the processing/observing instrument of a first embodiment of the present invention. This device comprises a processing ion beam irradiation system 1, an observing ion beam irradiation system 2, a detector 26 for detecting secondary electrons emitted from a specimen, a control unit 3 for controlling each of the irradiation systems 1 and 2, a switching means 4 for switching between the irradiation systems 1, 2, an exhaust pump 5 for exhausting air from each of columns 1a, 2a, a specimen holder 30 for holding the specimen, a display 3a for displaying a detection result obtained by the detector 26, and an input device 3b for receiving input signals such as the switching instruction.

The specimen holder 30 comprises a specimen stage 6 for holding the specimen 7 and displacing it in a horizontal direction, a specimen chamber 17 for maintaining the atmosphere around the specimen under vacuum, a mirror 19, and a laser position measurement device 20. In this embodiment, the specimen 7 is placed on the stage 6. A beam irradiation axis 102 of the observing ion beam irradiation system 2 is tilted at an angle of 60 to a beam irradiation axis 101 of the processing ion beam irradiation system 1. Located at a point of intersection of the axes 101, 102 is a surface of the specimen 7 placed on the specimen stage 6. An angle between the specimen surface to be processed and the irradiation axis 101 of the processing ion beam irradiation system 1 is 90°, and an angle between the specimen surface to be processed and the irradiation axis 102 of the observing ion beam irradiation system 2 is about 30°. These angles are, of course, not limiting, and other angles could be used if desired.

In this embodiment, a means for changing an angle of the specimen is not needed, because a processed cross section can be observed without tilting the specimen stage 6. Thus, the present embodiment can save the time to tilt the stage for observation and the time to bring it back to its horizontal position for processing, thus not causing a position shift accompanied by the tilting. This leads to a significant improvement in efficiency. Additionally, the mirror 19 and the laser position measurement device 20 can measure a position of the specimen by the order of submicron. The embodiment need not tilt the specimen, which enables a high precision measurement device to be incorporated.

As an example, the specimen 7 can be a wafer 300 mm (i.e. 12 inches) in diameter. In this embodiment, the specimen is not tilted, which makes it possible to reduce the height H of the specimen chamber 17 to less than 0.3 times that of the specimen chamber in the conventional device in which the specimen had to be tilted. Also, the whole processing/observing instrument according to the present embodiment is only approximately half as heavy as the conventional device.

The processing ion beam irradiation system 1 comprises the column 1a, an ion source 8, an extraction electrode 9, an ion beam focusing means (e.g., a condenser lens used as a focusing lens) 10, an objective lens 16, a blanking electrode 12, a beam restriction diaphragm 13, a precise restriction diaphragm mechanism 14 and an aligner mechanism 11, and an ion beam deflecting means (e.g., a scanning electrode 15).

Optionally, a mode switching means can be provided in the processing ion beam irradiation system 1 for switching the processing ion beam irradiation system 1 between a processing mode and a rough observing mode. As noted previously, if this arrangement is used, the processing ion beam irradiation system 1 will carry out initial rough observations, while the observing ion beam irradiation system 2 will carry out fine observations. Accordingly, the following discussion will describe the operation of the arrangement of FIG. 1 in a manner in which the processing ion beam irradiation system 1 will have both a processing mode and a rough observing mode. It is to be understood, however, that the system of FIG. 1 could also operate in a manner in which the processing ion beam irradiation system 2 would handle all observing operations.

The present embodiment employs a Ga liquid metal ion source as the ion source 8, and Ga ions are emitted by an electric field applied between the ion source 8 and the extraction electrode 9. These ions are focused onto the specimen 7 by means of the condenser lens 10 and the objective lens 16.

As noted above, the processing ion beam irradiation system 1 can perform a rough observation on the specimen as well as process it by changing an operation mode of the lenses (i.e. by changing a probe current and the lens voltage). For this purpose, under control by the control unit 3, a mode switching means (not shown) changes the operation mode by changing a diameter of the beam restriction diaphragm 13, the lens voltage and so on in accordance with an instruction transmitted from the control unit 3.

The rough observing mode of the processing ion beam irradiation system 1 is a mode which operates the objective lens 16 only, and it operates with the probe current of 100 pA or less and the probe diameter (i.e., the diameter of the beam on the substrate) of 100 nm or less. The small current in this mode enables a comparatively small quantity of specimen to be sputtered, and the narrow beam diameter makes the observation easier.

The processing mode of the processing ion beam irradiation system 1, on the other hand, is a mode which has the ion beam focused between the condenser lens 10 and the objective lens 16. This mode operates with the probe current 10 nA or more, thus being suited to process the specimen surface. When carrying out the processing, the following procedure of using the processing ion beam irradiation system simplifies an arrangement of the position to be processed: (1) obtaining an image in the vicinity of the position to be processed on the specimen surface with the rough observing mode; (2) deciding based on this image, the position actually to be processed; and (3) carrying out the processing by changing into the processing mode. Subsequently, fine observation can be carried out by the observing ion beam irradiation system 2, as well be discussed later.

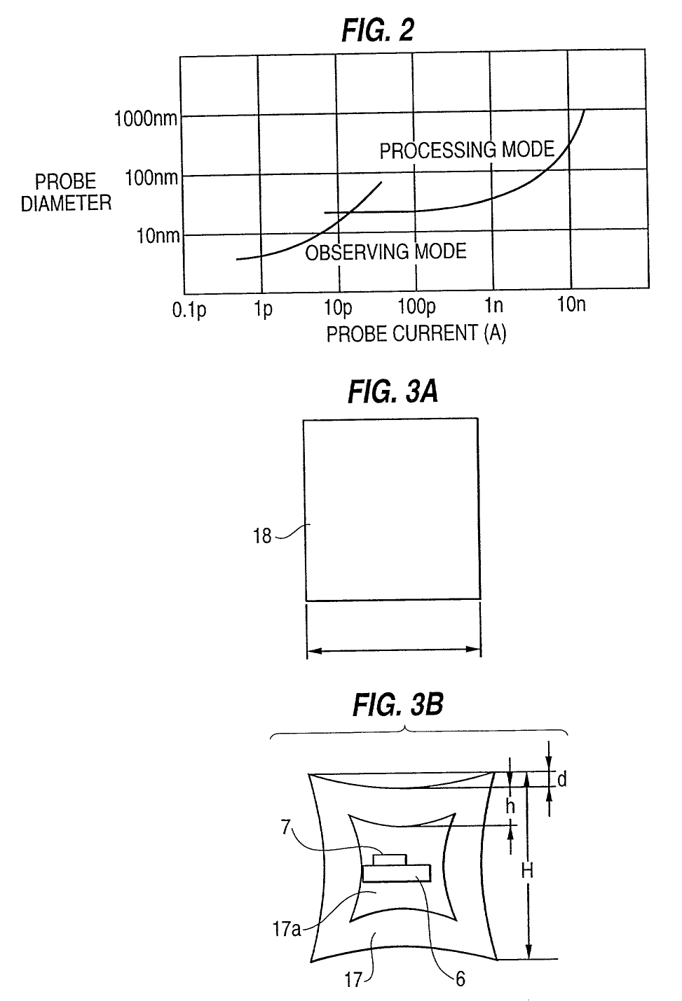

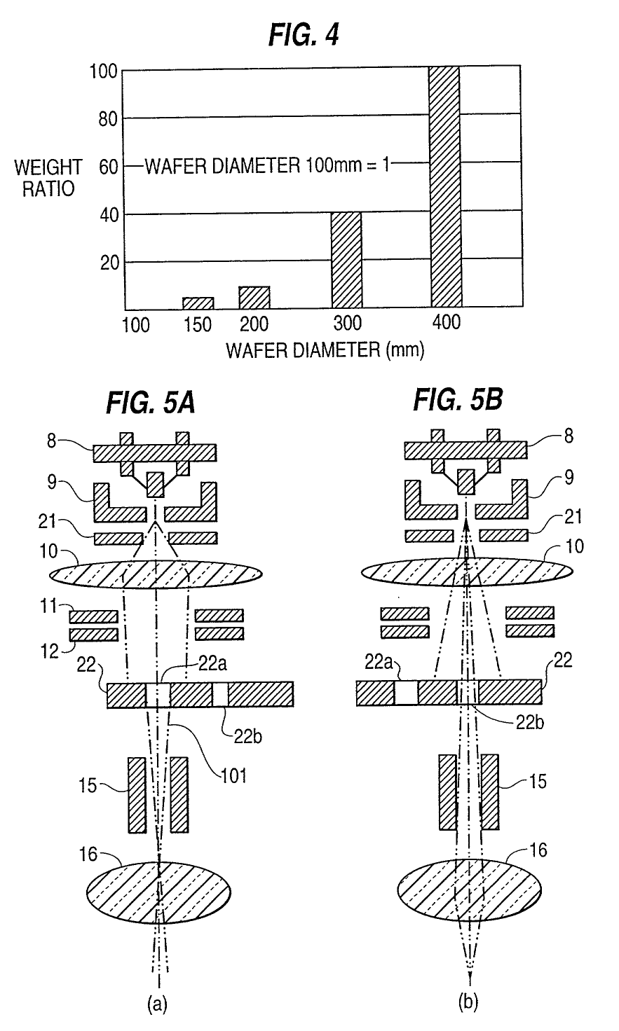

The probe diameter and the probe current on the specimen, when the lens diameter is left unchanged, are decided by a beam aperture angle (i.e., a diameter of the beam restriction diaphragm 13) and the lens mode (i.e., lens voltage). In this embodiment, FIG. 2 shows the relationship between a beam current (which is proportional to the diameter of the restriction diaphragm) and a beam diameter for the processing mode of the processing ion beam irradiation system 1 and for the observing modes of either of the processing ion beam irradiation system 1 or the observing ion beam irradiation system 2.

In the case of the processing mode of the processing ion beam irradiation system 1, the relationship between a processing rate and a fineness in processing often causes the beam to be switched among a coarse processing beam, an intermediate processing beam and a finishing processing beam. If, in the cases of these switchings and of the switching between the observing beam and the processing one, the axes of the beams do not exactly coincide with each other, the position which has been processed will shift from the position originally designated. Thus, the processing/observing instrument of the present embodiment is designed so that the beam irradiation axes will not shift in the case of such switching. Namely, the processing ion beam irradiation system 1 comprises a precise restriction diaphragm mechanism 14, the aligner mechanism 11 and so on, to prevent the irradiation axes from shifting in the case of the switching between the processing beam and the rough observing beam in the processing ion beam irradiation system 1.

The observing ion beam irradiation system 2 comprises an ion beam irradiation column 2a, an ion source 8, an extraction electrode 9, an ion beam focusing means (e.g., an objective lens 16, a blanking electrode 12, and a beam restriction diaphragm 13), and an ion beam deflecting means (e.g., a scanning electrode 15). A detector 26 detects secondary charged particles (in this embodiment, secondary electrons) emitted from the specimen by the ion beam irradiation. The detector 26 is coupled to the control unit 3 which includes means for converting the detection signals from the detector 26 into image signals for displaying the SIM image on a display screen 3A. A SIM image, which, compared with a SEM image, is prominent in a difference in a secondary electron signal quantity produced due to a difference in an atomic number, is suitable for observing the cross section of a composite material used for LSI and so on.

The observing ion beam irradiation system 2 does not need a focusing lens 10, a mode switching means, a precise restriction diaphragm mechanism 14 and an aligner mechanism, because it is used as an observation mode only. Concerning the other components, however, there are many devices common to both the processing ion beam irradiation system 1 and the observing ion beam irradiation system 2. In this embodiment, accordingly, the two irradiation systems 1, 2 use in common almost all control units except one regarding the mode switching between a processing mode and an observing mode. Also, a common ion pump 5 exhausts air from the columns 1a, 2a of the two irradiation system 1, 2.

Because the observing mode does not change the probe current very much, it has little need of changing the diameter of the beam restriction diaphragm. The probe current is usually taken as 1 through 2 pA. In this case, the probe diameter is 10 nm or less, which is small and suitable enough to observe the cross section processed by the processing ion beam irradiation system 1.

As shown in FIG. 1, the control unit 3 is an information processing device which comprises a main storage unit 31, a central processing unit (CPU) 32, and an external storage unit 33. The control unit 3, following an instruction introduced through an input unit 3b and by way of a switching means 4, transmits to the processing ion beam irradiation system 1 a control signal for changing the operation mode of the processing ion beam irradiation system 1 from a processing mode to a rough observation mode or a control signal for controlling the ion beam deflecting means 15. Also, the unit 3, responding to an instruction introduced through the input unit 3b, transmits to the switching means 4 a control signal for instructing the switching between the processing ion beam irradiation system 1 and the observing ion beam irradiation system 2.

Additionally, in the present embodiment, the control unit 3 can be an information processing device in which the above-stated control signals are generated in such a manner that the program stored beforehand in the external storage unit 33 is read into the main storage unit 31, and the CPU 32 performs the instruction included in the program stored in the main storage unit 31. The present invention, however, is not restricted to this. The control unit 3 may be a general-purpose processor which performs the above-mentioned program stored beforehand, or a specific hardware including a hard-wired logic.

The present second embodiment employs, as the processing ion beam irradiation system 1, a FIB device which can be switched between a projection beam mode and a scanning beam mode by changing the lens condition and the mask. The other components are substantially the same as the first embodiment. FIGS. 5A and 5B show the processing ion beam irradiation system 1 according to the present second embodiment, noting that the other elements of the second embodiment are not shown since they are substantially identical to the first embodiment shown in FIG. 1. Referring to FIGS. 5A and 5B, the irradiation system 1 comprises, inside a column 1a (not shown), an ion source 8, an extraction electrode 9, an aperture 21, a mask and diaphragm 22, an ion beam focusing means (e.g., a condenser lens used as a focusing lens) 10, an aligner mechanism 11, a blanking electrode 12, the mask and diaphragm 22 and an objective lens 16), an ion beam deflecting means (e.g., a scanning electrode 15), and a mode switching means (not shown in FIGS. 5A and 5B to switch between the projection beam mode and the scanning beam mode, as will be discussed below). The mask and diaphragm 22 comprises a through hole 22a for a pattern to be projected, and a through hole 22b used as a beam restriction diaphragm.

In the projection beam mode (normally only used during a processing mode), as is shown in FIG. 5A, the focusing lens 10 is operated as a projection lens to irradiate the ions through the through hole 22a in the mask and diaphragm 22 for the pattern to be projected. The objective lens 16 operating as a projection lens projects the mask pattern created by the hole 22a onto the specimen at a definite reduction ratio, thereby sputtering the projected part on the surface of the specimen 7. In this case, compared with the focused ion beam, processing at a higher current density is possible, which improves the processing rate. Furthermore, because it is difficult to prepare a large number of the through holes in the mask, the projection beam is effective in repeating the same configuration processing many times.

In the scanning mode (which can be used for either processing or observing), as is shown in FIG. 5B, the through hole 22b for providing a beam restriction diaphragm is employed. In the present embodiment, similar to the case with the observing modes of the first embodiment, the processing/observing instrument, when an ion image of the position to be processed is displayed in the observing mode in this scanning mode, receives, on this image, an input of the designation of the position by way of an input device 3b, and, by controlling the scanning electrode 15, deflects the ion beam so that the designated position will coincide with the position actually being processed. Thus, as a method of correcting an axis shift between the projection mode and the focused mode, a control unit 3 stores the voltage of the aligner/stigma electrode 11 beforehand to realize the value according to the mode change.

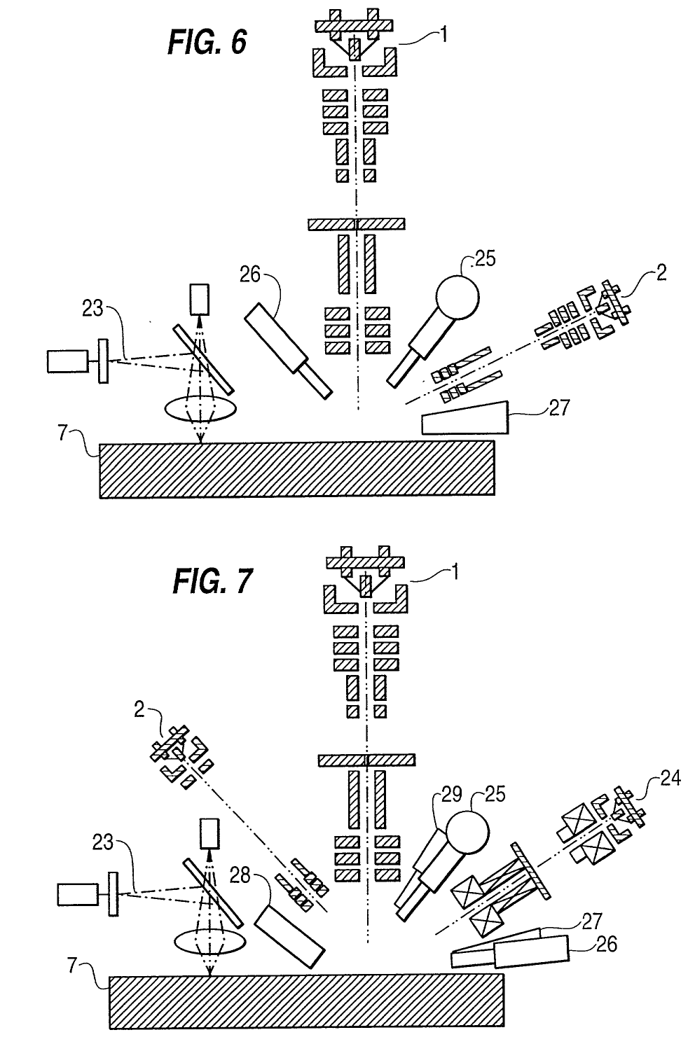

The present third embodiment is an embodiment of a processing/observing instrument in which the processing/observing instrument of the first embodiment further includes, as a second observing means, a confocal scanning laser microscope and a mass spectrometer. FIG. 6 shows the processing/observing instrument according to this present third embodiment.

The processing/observing instrument according to the present embodiment comprises, as in the case with the Example 1, a processing ion beam irradiation system 1, an observing ion beam irradiation system 2, a secondary electron detector 26, a control unit 3, a switching means 4, an exhaust pump 5, a specimen holder, a display 3a, in addition to an input device 3b, a confocal scanning laser microscope 23 (an optical microscope) for observing the specimen surface with a light beam, a gas supplying means 25 for supplying a gas for the inside of a specimen chamber 17, and a mass spectrometer 27 for mass-analyzing secondary ions emitted by an irradiation of the ion beam. With this arrangement, the confocal scanning laser microscope 23 effectively operates as a light beam arrangement. Incidentally, it is noted that elements of this embodiment which are substantially identical to the embodiment of FIG. 1 are not shown in FIG. 6 for drawing simplification.

When a specimen 7 is a flattened semiconductor device, an image obtained by the ion beam (i.e., SIM image) often fails to confirm a position of wiring in the lower layer. In this case, as is shown in the present embodiment, employing a beam means capable of transmitting through a protection film, such as the confocal scanning laser microscope 23, often makes it possible to confirm the position of wiring in the lower layer. The device of the embodiment, consequently, enables a semiconductor wafer to be processed, repeating the following steps:

(1) The control unit 3 operates in such manner that a state of the specimen is displayed using the confocal scanning laser microscope 23, after which the control unit 3 receives an input of the position to be processed by way of the input device 3b, and uses the main storage unit 31 to store the input of the position precisely.

(2) The control unit 3, by adding to the above stored position to be processed to a magnitude measured beforehand of the shift between an optical axis of the beam means 1 and that of the confocal scanning microscope 23, determines the position to actually be processed, and controls a stage 6 to displace the specimen 7 to the position to actually be processed, thereby processing the specimen surface, using the processing focused ion beam irradiation system 1.

(3) The control unit 3 operates to switch the irradiation systems by way of the switching means 4, to irradiate the ion beam to the processed cross section using the observing focused ion beam irradiation system 2, and to display on the display 3a an output from the detector 26 which has detected the secondary electrons, thereby displaying the SIM image of the processed cross section.

In this embodiment, since the sample 7 need not be tilted, the high stage accuracy enables the processing in the step (2) within an accuracy of 1 μm or less. When even higher accuracy of the position to be processed is required, the processing should be carried out as follows: after deciding the position to be processed by using the step (1), a mark is made in the vicinity of the position by means of the processing focused ion beam irradiation system 1, the processing again is returned to the step (1), the position measurement difference is measured between the mark on the upper layer and the wiring in the lower layer by using a measurement arrangement which the scanning laser microscope has, finally, the processing is returned to the step (2) again, and based on the position measurement difference, the position to actually be processed is determined by using a measurement means on the SIM image.

The instrument according to the present embodiment comprises a quadruple mass spectrometer 27 as a means for analyzing the portion to be observed. The quadruple mass spectrometer 27 analyzes secondary ions which are generated by an irradiation (fixed or scanning) of the beam to the position with the use of the observing focused ion beam irradiation system 2, thereby making it possible to mass-analyze the portion to be observed. In the present embodiment, because the stage is not tilted, its analysis efficiency has been improved in the mass analysis tremendously, compared with the conventional device that had to tilt the specimen.

Moreover, the generation efficiency of the secondary ions is improved by supplying an oxygen gas or an iodine gas into the specimen chamber. When performing the mass analysis, consequently, in the present embodiment, a gas introducing means 25 is disposed to supply an oxygen or an iodine gas into the specimen chamber 17. In this embodiment, the mass analysis of a semiconductor silicon wafer was performed with Ga as an ion in the observing focused ion beam irradiation system 2 using a high voltage acceleration voltage such as 30 kV. As a result, the introduction of an oxygen gas into the specimen chamber 17 has enhanced the generation efficiency of Si ions 40 times compared with the case otherwise.

Also, if a metal depositing gas or an insulator depositing gas is supplied by the gas supplying means 25, an exposure of the focused ion beam by the processing ion beam irradiation system 1 enables a beam assisted deposition to be performed. If a halogen, its gaseous compounds or water vapor is introduced into the specimen chamber 17 by the gas supplying means 25, an irradiation of the focused ion beam by the processing ion beam irradiation system 1 enables a beam assisted etching to be performed.

The present fourth embodiment shown in FIG. 7 is an embodiment of a processing/observing instrument in which the processing/observing instrument according to third embodiment further comprises a scanning electron microscope (SEM) 24, an inert gas element ion supplying means 29, and an x-ray detector 28. According to the present embodiment, characteristic x-rays can be detected by the x-ray detector 28 and the processed cross section can be observed by the SEM 24.

In the case of the observation with the SEM 24, due to a few factors, such as a redeposition of the shavings produced by the processing or a mixing on the specimen surface caused by the processing ions, the specimen cannot be satisfactorily observed by the electron beam. In such a case, the position to be processed is irradiated by the inert gas ions with the inert gas (argon in this case) ion supplying means 29 so that an undesired layer is removed. Thus a clear SEM image can be obtained. This also makes it unnecessary to tilt the specimen in order to remove the undesired layer. Consequently, the employment of the inert gas ion supplying means 29 permits observation of the cross section without tilting the specimen even when using the SEM 24.

As described above, the present embodiment comprises many kinds of processing, observing and analyzing components. The present invention is the first to realize such a complicated combination. A conventional specimen-tilting method requires enough space to tilt the specimen, leading to a reduction of the space in which each component can be incorporated. This, in carrying out the design of the processing/observing instrument, has made it practically impossible to realize such combination with conventional methods.

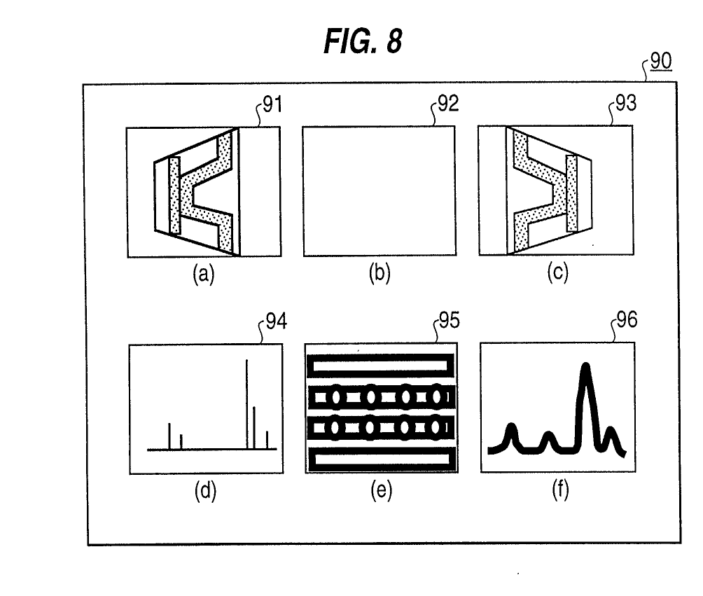

FIG. 8 shows examples of the images displayed in the present fourth embodiment of FIG. 7. A control unit 3 can, within a single display screen 90 of a display 3a, display a SIM image 91 of the processed cross section by means of the beam irradiated by an observing ion beam irradiation system 2, a SIM image 92 of the sample surface (in this case, the sample is a flat device, which makes the image all over white) by means of the beam irradiated by a processing ion beam irradiation system 1, a SEM image 93 of the processed cross section by means of the SEM 24, a mass spectrum 94 by means of a mass spectrometer 27, a microscope image 95 of the specimen surface by means of a scanning laser microscope image 23, and an x-ray spectrum 96 by means of the x-ray detector 28. This is possible because the control unit 3 comprises a means which stores the information for displaying these images in a main storage unit 31, edits it and outputs the information to the display 3a. According to the present embodiment, a variety of information which cannot be obtained by the conventional device can be displayed in the same screen.

It is noted that the above discussion emphasizes the significant advantages of the present invention in not requiring the specimen surface to be tilted for observation. On the other hand, an alternative arrangement using the present invention could implement a transmission electron microscope (TEM) to observe the specimen surface. In such a case, it would be allowable to slightly tilt the surface of the process specimen by an angle of ±5° to a plane, the normal of which is the irradiation access of the processing ion beam irradiation system. Although allowing for this small degree of tilting would slightly increase the size of the specimen chamber, the system would still be much smaller and lighter than that which would be required for processing and observing large diameter wafers using conventional techniques.

While the present invention has been particularly shown and described in reference to preferred embodiments thereof, it will be understood by those skilled in the art that changes in form and details may be made therein without departing from the spirit and scope of the invention.

What is claimed is:

1. A processing/observing instrument for processing and observing a specimen using a focused ion beam, comprising:
a processing ion beam irradiation system which irradiates a focused ion beam onto a specimen to process a surface of said specimen;
an observing ion beam irradiation system which irradiates a focused ion beam onto the processed surface of the specimen to make the specimen emit secondary charged particles;
a specimen holder which holds said specimen; and
a detector to detect the secondary charged particles emitted from the specimen and to produce an output indicative of a result of the detection;
wherein said processing ion beam irradiation system and said observing ion beam irradiation system are disposed in such a manner that an irradiation axis of said processing ion beam irradiation system forms an angle other than 0° to the irradiation axis of said observing ion beam irradiation system.

2. A processing/observing instrument according to claim 1, wherein said specimen holder holds said specimen so that said surface is at a point of intersection of said irradiation axis of said processing ion beam irradiation system and said irradiation axis of said observing ion beam irradiation system.

3. A processing/observing instrument according to claim 1, wherein said processing ion beam irradiation system and said observing ion beam irradiation system comprise, respectively,
an ion beam irradiation column the inside of which can be maintained in vacuum,
an ion source disposed in said column which generates an ion beam, and
an ion beam focusing unit disposed in said column which focuses said ion beam emitted from the ion source.

4. A processing/observing instrument according to claim 2, wherein said specimen holder holds the specimen in such a manner that said specimen surface to be processed forms an angle of less than ±5° to a plane the normal of which is said irradiation axis of said processing ion beam irradiation system.

5. A processing/observing instrument according to claim 3, wherein said processing ion beam irradiation system further comprises a mode switch which, in accordance with an instruction provided from outside the instrument, switches the operation mode between a processing mode for performing said processing and a rough observing mode for irradiating a focused ion beam onto the specimen to make the specimen emit secondary charged particles.

6. A processing/observing instrument according to claim 3, further comprising a common control unit which controls at least a part of said ion source and said ion beam focusing unit in said processing ion beam irradiation system and in said observing ion beam irradiation system, and a switch which, in accordance with an instruction from outside the instrument, selects either said processing ion beam irradiation system or said observing ion beam irradiation system.

7. A processing/observing instrument for processing and observing a specimen using a focused ion beam, comprising:
a processing ion beam irradiation system for irradiating a focused ion beam onto a specimen to process a surface of the specimen;
an observing ion beam irradiation system for irradiating a focused ion beam to said processed surface of the specimen to make said specimen emit secondary charged particles;
a specimen holder for holding the specimen so that said surface is at a point of intersection of an irradiation axis of said processing ion beam irradiation system and on irradiation axis of said observing ion beam irradiation system wherein an angle greater than 0° is formed between said irradiation axis of said processing ion beam irradiation system and said irradiation axis of said observing ion beam irradiation system;
a detector for detecting the secondary charged particles emitted from the specimen and producing an output indicative of a result of the detection; and
a display for displaying the output from said detector,
wherein said processing ion beam irradiation system and said observing ion beam irradiation system comprise, respectively,
an ion beam irradiation column the inside of which can be maintained in vacuum,
an ion source disposed in said column for generating an ion beam, and
an ion beam focusing means disposed in said column for focusing the ion beam emitted from the ion source.

8. The processing/observing instrument according to claim 7, wherein said specimen holder supports the specimen in such a manner that said specimen surface to be processed forms an angle of less than ±5° to a plane the normal of which is said irradiation axis of said processing ion beam irradiation system.

9. The processing/observing instrument according to claim 7, wherein said specimen holder comprises an xy stage which can displace the specimen 300 mm or further in both an x-axis direction and a y-axis direction in a plane the normal of which is the irradiation axis of said processing ion beam irradiation system.

10. The processing/observing instrument according to claim 7, wherein said processing ion beam irradiation system further comprises a mode switching means which, in accordance with an instruction introduced from outside the instrument, switches the operation mode between a processing mode for performing said processing and a rough observing mode for irradiating a focused ion beam onto the specimen to make the specimen emit secondary charged particles.

11. The processing/observing instrument according to claim 10, wherein said mode switching means comprises
a means which, when said processing mode is instructed, generates a probe current having an amplitude of 3 nA or more, and
a means which, when said observing mode is instructed, generates a probe current having an amplitude of 30 pA or less.

12. The processing/observing instrument according to claim 7, further comprising a common control unit which controls at least a part of said ion source and said ion beam focusing means in said processing ion beam irradiation system and said observing ion beam irradiation system, and a switching means which, in accordance with an instruction from outside the instrument, selects either said processing ion beam irradiation system or said observing ion beam irradiation system.

13. The processing/observing instrument according to claim 7, further comprising:
a second observing means for producing an output of the information indicating a surface condition of said specimen held by said specimen holder, wherein said second observing means is at least one of a scanning electron microscope and an optical microscope.

14. The processing/observing instrument according to claim 13, wherein said display comprises a plural data displaying means which, on a same screen, displays an output of said second observing means as well as that of said detector.

15. The processing/observing instrument according to claim 13, wherein said second observing means is a scanning laser microscope.

16. The processing/observing instrument according to claim 7, further comprising a means for supplying a gas for the inside of said ion beam irradiation column in said processing ion beam irradiation system.

17. The processing/observing instrument according to claim 7, wherein said observing ion beam irradiation system further comprises a means for mass-analyzing secondary ions included in said secondary charged particles.

18. A processing/observing instrument according to claim 1, wherein the angle between the irradiation axis of the processing ion beam irradiation system and the irradiation axis of the observing ion beam irradiation system is 60°.

19. A processing/observing instrument according to claim 7, wherein the angle between the irradiation axis of the processing ion beam irradiation system and the irradiation axis of the observing ion beam irradiation system is 60°.
